(19) United States
(12) Patent Application Publication
(10) Pub. No.: US 2008/0119855 A1
(43) Pub. Date: May 22, 2008

(54) Orthopedic fixation system

(75) Inventors: Roland Alois Hoegerle, Tuttlingen (DE); Frank Altermann, Tuttlingen (DE); Hans-Werner Stedtfeld, Nuernberg (DE); Martyn Parker, Peterborough (GB)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/891,306

(22) Filed: Aug. 9, 2007

Related U.S. Application Data
(63) Continuation of application No. PCT/EP2006/001304, filed on Feb. 14, 2006.

(30) Foreign Application Priority Data
Feb. 19, 2005 (DE) 10 2005 007 674.2

(51) Int. Cl. A61B 17/58; A61B 17/56

(52) U.S. Cl. 606/65; 606/60; 606/301

(57) ABSTRACT

In order to simplify the implantation of an orthopedic fixation system for securing a first bone fragment to a second bone fragment with a support screw which can be screwed into the first bone fragment by means of a bone thread, with a support sleeve surrounding the support screw and guiding this so as to be freely displaceable in longitudinal direction and with a bone plate which has at least one threaded bore, can be secured to the second bone fragment and into the threaded bore of which the support sleeve can be screwed by means of an external thread, it is suggested that the bone thread, on the one hand, and the threaded bore and the external thread, on the other hand, have the same pitch of the thread turns and that the bone thread have one thread turn and the threaded bore as well as the external thread have at least two thread turns located next to one another with a correspondingly smaller width of each thread turn.

This application is a continuation of International application No. PCT/EP2006/001304 filed on Feb. 14, 2006.

The present disclosure relates to the subject matter disclosed in International application No. PCT/EP2006/001304 of Feb. 14, 2006 and German application No. 10 2005 007 674.2 of Feb. 19, 2005, which are incorporated herein by reference in their entirety and for all purposes.

### BACKGROUND OF THE INVENTION

The invention relates to an orthopedic fixation system for securing a first bone fragment to a second bone fragment with a support screw which can be screwed into the first bone fragment by means of a bone thread, with a support sleeve surrounding the support screw and guiding this so as to be freely displaceable in longitudinal direction and with a bone plate which has at least one threaded bore, can be secured to the second bone fragment and into the threaded bore of which the support sleeve can be screwed by means of an external thread.

In order to fix two bone fragments relative to one another, for example, two bone fragments in the area of the femoral neck of a femur bone, it is known to screw a support screw into one of the two bone fragments, for example, the head of the femur and to guide this for longitudinal displacement in a support sleeve which is secured to the second bone fragment, i.e., for example, to the upper part of the femur such that the support screw is guided in the support sleeve but permits longitudinal displacement.

In the case of known orthopedic fixation systems of this type, the support sleeve is connected in one piece to a bone plate which can be secured to the second bone fragment (U.S. Pat. No. 2,702,543, U.S. Pat. No. 2,612,159). There are also fixation systems, with which the support sleeve can be pushed into the bone plate (U.S. Pat. No. 4,438,762) and, in the case of other systems, the support sleeve is screwed directly into the bone (U.S. Pat. No. 4,940,467).

Another possibility of securing the support sleeve on the bone plate can provide for the support sleeve to be screwed into the bone plate. Since the bone plate has only a slight thickness, all the bending forces must be transferred from the support sleeve to the bone plate in the area of the screw connection and it is, therefore, necessary to arrange a stable mechanical thread in this area.

On the other hand, the support screw is normally screwed into the bone with a so-called bone thread; in this respect, this is mostly a self-cutting thread with a relatively high pitch and relatively large spacing between adjacent thread turns. Separate operating steps are, therefore, necessary in order to screw the support screw into the first bone fragment and to screw the support sleeve into the bone plate.

The object of the invention is to improve an orthopedic fixation system of the generic type such that this system is easier to implant.

### SUMMARY OF THE INVENTION

This object is accomplished in accordance with the invention, in an orthopedic fixation system of the type described at the outset, in that the bone thread, on the one hand, and the threaded bore and the external thread, on the other hand, have the same pitch of the thread turns and that the bone thread has one thread turn and the internal threaded bore as well as the external thread have at least two thread turns located next to one another with a correspondingly smaller width of each thread turn.

As a result of threads with the same pitch being used, support screw and support sleeve can be screwed together into the first bone fragment and the bone plate, respectively, without their relative position being thereby altered in an axial direction. On the other hand, the support sleeve experiences a particularly good fixing in the internal threaded bore of the bone plate due to the fact that not only one thread turn is provided but rather at least two thread turns, preferably three thread turns, next to one another. The increase in the number of thread turns has, in addition, the advantage that there are several starting points for thread turns along the circumference of the support sleeve and so the external thread can already engage in the internal threaded bore after one rotation, the angle of which is considerably less than 360°; at the most this angle corresponds to the quotient resulting from a full rotation and the number of thread turns.

The bone plate can have several threaded bores for receiving support sleeves and so the operator is given several possibilities, following abutment of the bone plate on the second bone fragment, of implanting support sleeves and, therefore, support screws; where applicable, several support sleeves with support screws can be implanted next to one another.

It is favorable when the support sleeve has a circular cylindrical inner wall which abuts closely on the circular cylindrical outer wall of a shaft section of the support screw. As a result, the support screw is guided in the support sleeve on all sides.

It is particularly advantageous when the circular cylindrical inner wall and the circular cylindrical shaft section widen in a step-like manner towards the external thread of the support sleeve so that the resulting step forms a stop which limits the withdrawal movement of the support screw out of the support sleeve in the direction towards the bone thread.

The support screw preferably has a receiving opening for the form locking introduction of a tool at its end located opposite the bone thread; this may, for example, be a hexagonal recess. In addition, it is favorable when the support sleeve has at its external thread an enlargement in its inner wall for the form locking introduction of a rotary tool. This enlargement can also be designed as a hexagonal recess in the support sleeve.

The invention also relates to a rotary instrument for the simultaneous turning in or out of the support screw and the support sleeve. Such a rotary instrument is characterized by the fact that a first rotary tool which can be inserted into the receiving opening in a form locking manner and a second rotary tool which can be inserted into the enlargement in a form locking manner are arranged on an elongated shaft, which can be introduced into the support sleeve, at its free end, the second rotary tool being at a distance on the shaft from the first rotary tool. Such a rotary instrument therefore forms a non-rotatable connection not only with the support screw but also with the support sleeve during the introduction of the rotary tools into the receiving opening and the enlargement, respectively, and so during the rotation of the rotary instrument the support screw and the support sleeve are turned together, for example, in order to screw both parts together into the first bone fragment and the bone plate secured on the second bone fragment, respectively.

The following description of preferred embodiments of the invention serves to explain the invention in greater detail in conjunction with the drawings.

### BRIEF DESCRIPTION OF THE DRAWINGS

FIG. 1: shows a cross-sectional illustration through a femur bone in the area of the neck of the femur with a bone plate secured to the femur and two support sleeves screwed into this, each with a support screw;

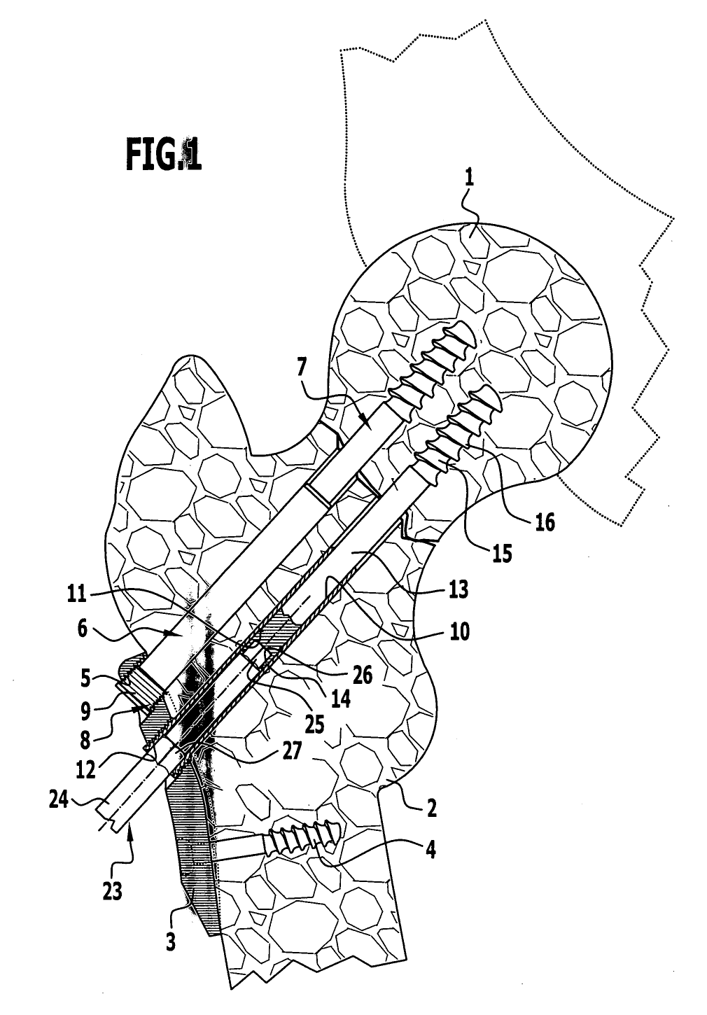

FIG. 2: shows a perspective view of the bone plate of FIG. 1 with a support sleeve cut away in longitudinal direction prior to being screwed into the bone plate and with a support screw in the support sleeve and

FIG. 3: shows enlarged illustrations of the bone thread of the support screw and the external thread of the support sleeve.

### DETAILED DESCRIPTION OF THE INVENTION

The orthopedic fixation system will be explained on the basis of the example of a fracture of the neck of the femur; in the case of such a fracture of the neck of the femur it is necessary to secure the part of the femur bearing the socket joint, i.e., a first bone fragment 1 relative to the remaining femur, i.e., a second bone fragment 2 and thereby make a certain movement between the two bone fragments possible in the direction of connection of the two bone fragments since this movement stimulates the formation of bone substance. The connection may not, therefore, be a rigid connection but rather be a connection, by means of which the bone fragments are guided relative to one another but can execute small movements in their direction of connection.

The orthopedic fixation system comprises a bone plate 3 which is secured to the outer side of the second bone fragment 2 on the side located opposite the first bone fragment 1. The bone plate 3 can be adapted to the outer contour of the second bone fragment 2 and is secured at this location in a conventional manner by means of one or several bone screws 4. In the embodiment illustrated, the bone plate 3 has in an upper part four internal threaded bores 5 which are located next to one another and are all inclined in the same way in relation to the contact surface of the bone plate 3; the longitudinal axes of the internal threaded bores 5 are inclined, for example, through 130° in relation to the contact surface of the bone plate 3 and extend parallel to one another.

The orthopedic fixation system comprises, in addition, at least one elongated support sleeve 6 and a support screw 7 associated with each support sleeve 6. The support sleeves 6 and the support screws 7 are of the same design when several support sleeves and support screws are used; only one example will therefore be explained in detail in the following.

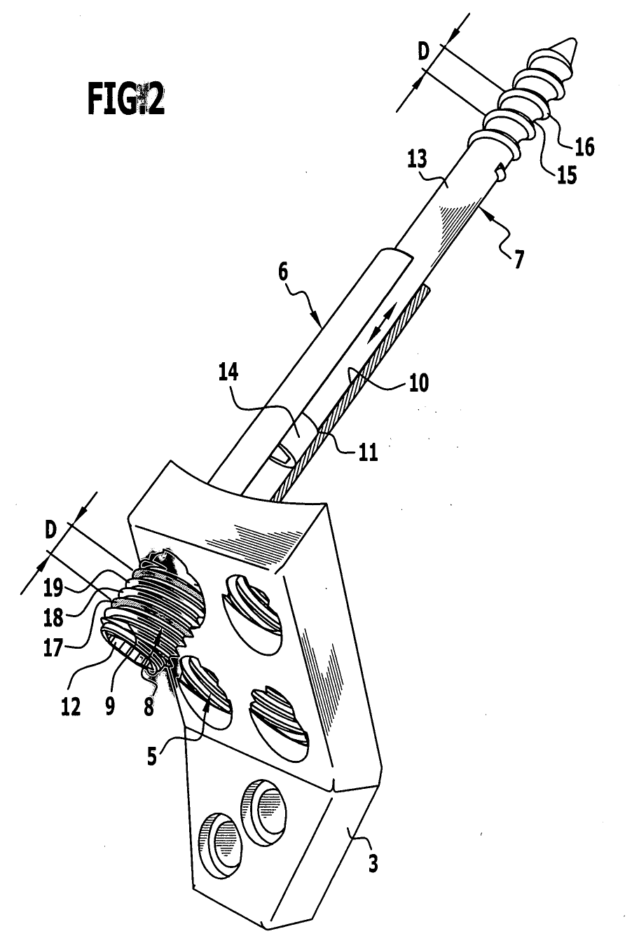

The support sleeve 6 has a screw-in area 8 at one end which is thickened towards the outside. In this screw-in area 8 it bears an external thread 9, with which the support sleeve 6 can be screwed into a respective internal threaded bore 5. The inner wall 10 of the support sleeve 6 is of a circular cylindrical design and widens approximately in the center of the support sleeve 6 in a direction towards the screw-in area 8 in a step-like manner so that an annular step 11 with a small height results at the transition of the circular cylindrical sections with different diameters (FIG. 2).

In the screw-in area 8, the inner wall 10 widens again and forms at this point an enlargement 12 with a cross section like a hexagonal recess; this enlargement 12 adjoins directly onto the end of the support sleeve 6 on the side of the external thread.

The support screw 7 is accommodated in the interior of the support sleeve 6 and this has a circular cylindrical shaft 13 with a circular cylindrical head 14 with a slightly greater diameter as well as a preferably self-cutting bone thread 15 at the end located opposite the head 14. The external diameter of the circular cylindrical shaft 13 corresponds to the internal diameter of the inner wall 10 in the more narrow part of the support sleeve 6 located opposite the screw-in area 8; the external diameter of the circular cylindrical head 14 corresponds to the diameter of the inner wall 10 in the area between the annular step 11 and the enlargement 12 so that the support screw 7 is guided in the support sleeve 6 so as to be freely displaceable in a longitudinal direction, wherein any withdrawal of the support screw 7 out of the support sleeve 6 is prevented in both directions, in the direction towards the bone thread 15 by the head 14 abutting on the annular step 11, in the opposite direction by the bone thread 15 abutting on the support sleeve 6.

The bone thread 15 has a thread turn 16 with a relatively high pitch, for example, with a pitch of 3 mm per full rotation.

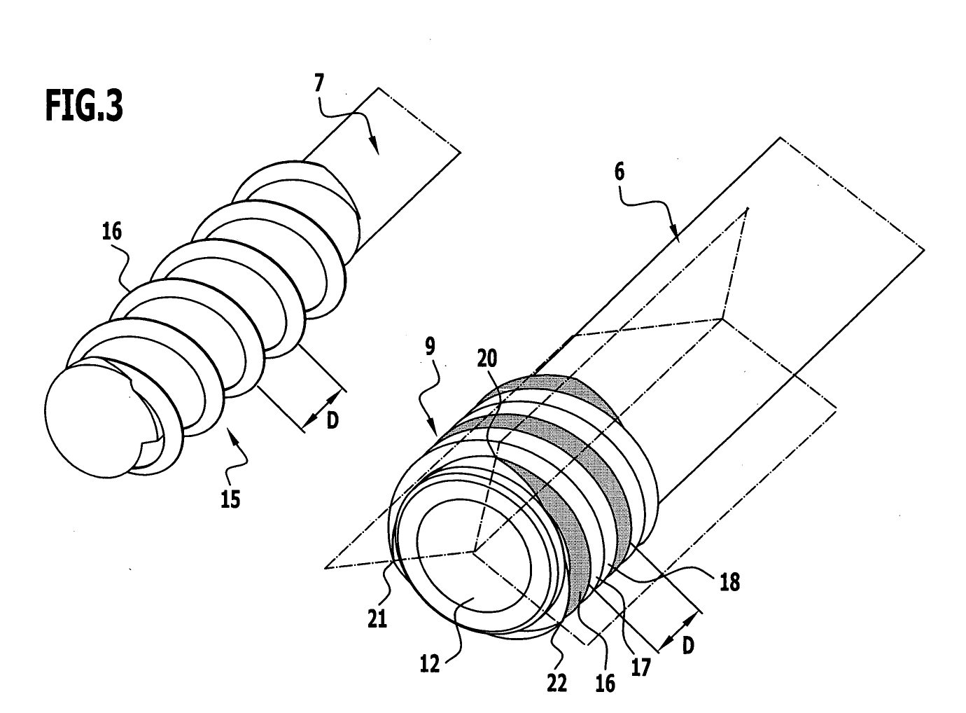

The external thread 9 also has the same high pitch but the external thread 9 comprises, in the embodiment illustrated in the drawings, three thread turns 17, 18, 19 which are located next to one another and one of which is illustrated in FIGS. 2 and 3 darkened for the purpose of clarification. The three thread turns 17, 18, 19 have a width which corresponds only to a third of the width of the thread turn 16; in addition, the beginnings 20, 21, 22 of the thread turns 17, 18, 19 are each offset in relation to one another through 120° in circumferential direction of the support sleeve 6 (FIG. 3). Adjacent sections of the same thread turn therefore have the same spacing D (FIG. 3), namely not only in the case of the bone thread 15 but also in the case of the external thread 9; the distance between adjacent thread turns is, however, in the case of the external thread only one third of the distance of that for the bone thread. As a result of the greater number of adjacent thread turns, the external thread 9 is screwed into the internal thread of the internal threaded bore 5, which is provided in the same way with three thread turns, so tightly that all the mechanical loads can easily be transferred in the connecting area.

A rotary instrument 23 is used to turn the support sleeve 6 and the support screw 7 in and out and only an elongated shaft 24 of this instrument, which bears a hexagonal projection 25 at its free end, is illustrated in the drawings. This fits in a form locking manner into a hexagonal recess 26 in the head 14 of the support screw 7.

In addition, the shaft 24 has at a distance to the hexagonal projection 25 a hexagonal projection 27 which fits in a form locking manner into the enlargement 12.

For the purpose of implanting the orthopedic fixation system, the bone plate 3 is secured first of all to the outer side of the second bone fragment 2 with the aid of a bone screw 4. Subsequently, the constructional unit consisting of support sleeve 6 and support screw 7 is inserted—where applicable after the introduction of a corresponding bore. For this purpose, the rotary instrument 23 with its shaft 24 is inserted into the support sleeve 6 such that the hexagonal projections 25 and 27 engage non-rotatably in the hexagonal recess 26 and the enlargement 12, respectively; this can be achieved in accordance with the dimensioning when the support screw 7 is pushed completely out of the support sleeve 6, when, therefore, the head 14 abuts on the annular step 11. The support sleeve 6 and the support screw 7 are turned together by the rotary instrument 23 while the support screw 7 with the bone thread 15 is screwed first of all into the first bone fragment 1. At the end of this screw-in process, the external thread 9 also reaches the area of the internal threaded bore 5; the external thread 9 thereby engages in the internal thread of the internal threaded bore 5 and, during further rotation, the support sleeve 6 is screwed with the external thread into the internal threaded bore as a result, namely with the same pitch, with which the bone screw 4 is screwed into the first bone fragment 1; the support sleeve 6 and the support screw 7 are therefore not moved relative one another in an axial direction during this screw-in process.

At its end on the side of the external thread, the support sleeve 6 can have an additional enlargement which determines the maximum screw-in depth. Following the screwing-in process, the rotary instrument 23 is drawn out of the support sleeve 6 and this is closed by means of a plug not illustrated in the drawings. In this installed position, the two bone fragments are secured relative to one another; it is possible for the first bone fragment to be moved slightly in the direction towards the second bone fragment when the bone is stressed so that the growth of the bone can be stimulated as a result of this slight movement and the additional stressing connected therewith in the contact area of the two bone fragments.

1. Orthopedic fixation system for securing a first bone fragment to a second bone fragment with a support screw adapted to be screwed into the first bone fragment by means of a bone thread, with a support sleeve surrounding the support screw and guiding this so as to be freely displaceable in longitudinal direction and with a bone plate having at least one threaded bore and being securable to the second bone fragment, the support sleeve being adapted to be screwed into the threaded bore of said bone plate by means of an external thread, wherein the bone thread, on the one hand, and the threaded bore and the external thread, on the other hand, have the same pitch of the thread turns and wherein the bone thread has one thread turn and the threaded bore as well as the external thread have at least two thread turns located next to one another with a correspondingly smaller width of each thread turn.

2. Fixation system as defined in claim 1, wherein the threaded bore and the external thread have three thread turns located next to one another.

3. Fixation system as defined in claim 1, wherein the bone plate has several threaded bores for receiving support sleeves.

4. Fixation system as defined in claim 1, wherein the support sleeve has a circular cylindrical inner wall abutting closely on the circular cylindrical outer wall of a shaft section of the support screw.

5. Fixation system as defined in claim 4, wherein the circular cylindrical inner wall and the circular cylindrical shaft section widen in a step-like manner towards the external thread end of the support sleeve so that the resulting step forms a stop limiting the withdrawal movement of the support screw out of the support sleeve in the direction towards the bone thread.

6. Fixation system as defined in claim 1, wherein the support screw has a receiving opening for the form locking introduction of a rotary tool at its end located opposite the bone thread.

7. Fixation system as defined in claim 1, wherein at its eternal thread end, the support sleeve has an enlargement in its inner wall for the form locking introduction of a rotary tool.

8. Rotary instrument for the simultaneous turning in or out of the support screw and the support sleeve of claim 1, wherein:
the support screw has a receiving opening for the form locking introduction of a rotary tool at its end located opposite the bone thread;
at its eternal thread end, the support sleeve has an enlargement in its inner wall for the form locking introduction of a rotary tool;
a first rotary tool insertable in a form locking manner into the receiving opening and a second rotary tool insertable in a form locking manner into the enlargement are arranged on an elongated shaft introducible into the support sleeve at its free end;
said second rotary tool being at a distance on the shaft in relation to the first rotary tool.
